(19) United States
(12) Patent Application Publication
(10) Pub. No.: US 2022/0213233 A1
(43) Pub. Date: Jul. 7, 2022

(54) CROSSLINKED POLYMER OF FUNCTIONALIZED HYALURONIC ACID AND ITS USE IN THE TREATMENT OF INFLAMMATORY STATES

(71) Applicant: JOINTHERAPEUTICS S.R.L., Como (IT)

(72) Inventors: Giulio Bianchini, Como (IT); Lanfranco Callegaro, Como (IT)

(73) Assignee: JOINTHERAPEUTICS S.R.L., Como (IT)

(21) Appl. No.: 17/604,772

(22) PCT Filed: Apr. 17, 2020
(86) PCT No.: PCT/IB2020/053643

§ 371 Date: Oct. 19, 2021

(30) Foreign Application Priority Data
Apr. 19, 2019 (IT) 102019000006146

(51) Int. Cl. C08B 37/08; A61K 45/06

(52) U.S. Cl. CPC C08B 37/0072; A61K 45/06

(57) ABSTRACT

A crosslinked polymer of functionalized hyaluronic acid, or derivative thereof, is disclosed, as well as a process for its preparation and its use as a biomaterial and as an ingredient in pharmaceutical compositions. The use of this crosslinked polymer in the treatment of disorders ascribable to altered galectin expression, is also disclosed.

### FIELD OF THE INVENTION

The present invention concerns a crosslinked polymer of functionalized hyaluronic acid, or a derivative thereof, as well as processes for the their preparation and use as biomaterials and as ingredients in pharmaceutical compositions.

### BACKGROUND ART

Receptor CD44 is a highly glycosylated transmembrane protein whose function is to bind the hyaluronic acid and other glycoproteins of the extracellular matrix. The bond between CD44 and hyaluronic acid does not serve solely for anchorage, but also allows the transduction of signals inside the cell. Another important family of receptors is that of the galectins, proteins defined by their bond specificity for β-galactoside sugars, and likewise N-acetyllactosamine, which can be bound to proteins through N-glycosylation or O-glycosylation. Current evidence indicates that galectins perform an important role in acute and chronic inflammatory responses and in various pathological processes. Recent studies show how the interaction between CD44 and galectins plays an active role in the regulation of cellular mechanisms (Immunity 2014, 41 (2), 270-282; The Journal of Immunology 2007, 179 (2), 1225-1235). Poor regulation of galectins, for example, overexpression, is typically encountered in inflammatory disorders, therefore correct regulation of these receptors can determine a marked reduction in the inflammatory cascade.

Some of the greatest problems linked to inhibitors/modulators of galectins during the study stage include the reduced capacity for target site recognition and permanence. Therefore, the object of the present invention is to provide a product which, through the simultaneous interaction with galectin receptors and receptor CD44 makes it possible to treat disorders ascribable to altered galectin expression, offering, at the same time a high level of acceptability from a medical and pharmaceutical perspective and in terms of improved permanence times at the target site.

### SUMMARY OF THE INVENTION

Said object has been achieved through a crosslinked polymer wherein functionalized hyaluronic acid, or a derivative thereof, is at least partially crosslinked as stated in Claim 1.

In a further aspect, the present invention concerns a process for the preparation of said crosslinked polymer.

In a still further aspect, the present invention concerns the use of crosslinked polymer in the treatment of disorders ascribable to altered galectin expression. Non-limiting examples of disorders concerned by over/under-regulation of these receptors are non-alcoholic steatohepatitis, plaque psoriasis, rheumatoid arthritis, osteoarthritis, neoplasms, adhesions, and dermal, pulmonary, renal, and cardiovascular fibrotic processes.

In a still further aspect, the present invention concerns the use of this crosslinked polymer as a biomaterial or scaffold for cellular growth, preferably in the treatment of orthopaedic disorders.

In an even further aspect, the present invention concerns the use of this crosslinked polymer as a biomaterial or scaffold for cellular growth, in plastic/cosmetic surgery, haemodialysis, cardiology, angiology, ophthalmology, otorhinolaryngology, dentistry, gynaecology, urology, dermatology, oncology and tissue repair. In a further aspect, the present invention regards a pharmaceutical composition comprising at least one crosslinked polymer and at least one pharmacologically active substance and/or at least one substance having, optionally, a biological function.

In a still further aspect, the present invention concerns the use of this pharmaceutical composition in the treatment of disorders ascribable to altered galectin expression. Non-limiting examples of disorders concerned by over/under-regulation of these receptors are non-alcoholic steatohepatitis, plaque psoriasis, rheumatoid arthritis, osteoarthritis, neoplasms, adhesions, and dermal, pulmonary, renal, and cardiovascular fibrotic processes.

In an even further aspect, the present invention concerns the use of this pharmaceutical composition in rheumatology, orthopaedics, oncology, plastic/cosmetic surgery, haemodialysis, cardiology, angiology, ophthalmology, otorhinolaryngology, dentistry, gynaecology, urology, dermatology, oncology, and tissue repair.

The characteristics and the advantages of the present invention will become clear in the following detailed description and the embodiments provided as non-limiting, illustrative examples.

### DETAILED DESCRIPTION OF THE INVENTION

The invention regards, therefore, a crosslinked polymer comprising functionalized hyaluronic acid, or a derivative thereof, comprising 10-90% of repeating units having the formula (I):

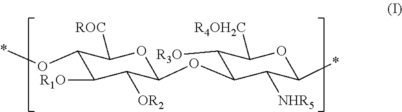

wherein
R₁, R₂, R₃, R₄ are, independently of one another, H, SO₃⁻, an acyl group derived from a carboxylic acid of the aliphatic, aromatic, arylaliphatic, cycloaliphatic, heterocyclic series, —CO—(CH₂)₂—COOY, where Y is a negative charge or H, and
R is Z(1) or Z(2), and R₅ is —CO—CH₃, H, SO₃⁻, an acyl group derived from a carboxylic acid of the aliphatic, aromatic, arylaliphatic, cycloaliphatic, heterocyclic series, an acyl group of hyaluronic acid,
where Z(1) is a moiety of formula (1):

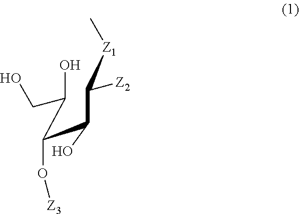

wherein Z₁ is —NR₆CH₂—, and R₆ is H or an aliphatic, aromatic, arylaliphatic, cycloaliphatic, heterocyclic group, substituted or non-substituted,
   Z₂ is —OH, or —NHCOCH₃,
   Z₃ is H, monosaccharide, disaccharide, or oligosaccharide, or Z(2) is a moiety of formula (2):

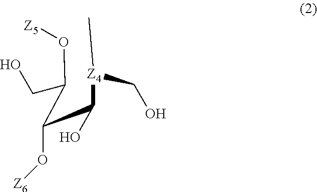

wherein Z₄ is —NR₆CH—, and R₆ is H or an aliphatic, aromatic, arylaliphatic, cycloaliphatic, heterocyclic group, substituted or non-substituted, Z₅ and Z₆ are, independently of each other, H, monosaccharide, disaccharide, or oligosaccharide,
   or
   R₅ is Z(3) or Z(4), and R is NR₆R₇, or an alcoholic group of the aliphatic, aromatic, arylaliphatic, cycloaliphatic, heterocyclic series, OH, O⁻, an alcoholic group of hyaluronic acid, an amino group of hyaluronic acid, and R₆, R₇ are, independently of each other, H or an aliphatic, aromatic, arylaliphatic, cycloaliphatic, heterocyclic group, substituted or non-substituted,
where Z(3) is a moiety of formula (3):

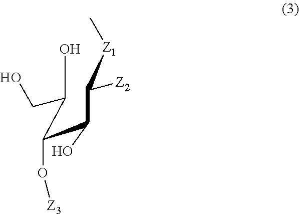

wherein Z₁ is —CH₂— or —CO—,
Z₂ is —OH, or —NHCOCH₃,
Z₃ is H, monosaccharide, disaccharide, or oligosaccharide, or Z(4) is a moiety of formula (4):

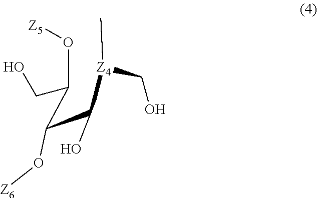

wherein Z₄ is —CH—,
Z₅ and Z₆ are, independently of one another, H, monosaccharide, disaccharide, or oligosaccharide, or

### R is Z(1) or Z(2), and R₅ is Z(3) or Z(4),

said functionalized hyaluronic acid, or derivative thereof, being at least partially crosslinked directly by ester bond or lactone bond between carboxyl groups and hydroxyl groups in the same chain of functionalized hyaluronic acid, or a derivative thereof, and/or between carboxyl groups and hydroxyl groups in different chains, or being at least partially crosslinked indirectly by a spacer moiety forming ester bonds with the carboxyl groups and/or ether bonds with the hydroxyl groups and/or amide bonds with the carboxyl groups, said spacer moiety being a biscarbodiimidic moiety or a bisvinylsulfonic moiety or an epoxy moiety deriving from bi- or polyfunctional epoxide selected from C2-C20 aliphatic epoxides, their halogenhydrons, epialogenhydrins, and halides, or a combination thereof.

The crosslinked polymer, as described above, has proved to be particularly suitable for therapeutic use in disorders ascribable to altered galectin expression, through the simultaneous interaction with galectin receptors and receptor CD44.

Furthermore, it has shown a high level of acceptability from a medical and pharmaceutical perspective and in terms of improved permanence times at the target site, since it features greater resistance to enzymatic breakdown, in addition to improved mechanical and physico-chemical properties.

Preferably, in the crosslinked polymer of the invention, said functionalized hyaluronic acid, or derivative thereof, comprises 10-60% of repeating units having the formula (I). The carboxyl groups and the hydroxyl groups of the functionalized hyaluronic acid, or derivative thereof, not involved in the crosslinking can, optionally, be salified, for example with cations of sodium, potassium, calcium, magnesium, ammonium or mixtures thereof.

In some embodiments, in the crosslinked polymer of the invention, 20-70% of the carboxyl groups and of the hydroxyl groups of the functionalized hyaluronic acid, or derivative thereof, not involved in the crosslinking are salified.

Preferably, 5-40% of the bonds between the spacer moiety and the functionalized hyaluronic acid, or derivative thereof, are ester bonds, more preferably 10-30%.

In first embodiments, in 20-60% of the repeating units having the formula (I) present in the crosslinked polymer, R is Z(1) or Z(2), more preferably in 30-50%. Preferably, in these first embodiments, R₅ is —COCH₃.

In second embodiments, in 5-30% of the repeating units having the formula (I) present in the crosslinked polymer, R₅ is Z(3) or Z(4), more preferably in 10-20%. Preferably, in these second embodiments, 10-20% of R₅ is Z(3) and 90-80% of R₅ is —COCH₃, while R is O—.

In further embodiments, the crosslinked polymer of the present invention comprises both repeating units having the formula (I) of said first embodiments and repeating units having the formula (I) of said second embodiments.

As stated above, the crosslinking of the polymer of the invention can take place directly, i.e. by intramolecular reaction and/or intermolecular reaction between free carboxylic and/or hydroxylic functional groups of the functionalized hyaluronic acid, or derivative thereof, or indirectly, i.e. by intramolecular reaction and/or intermolecular reaction by a spacer moiety between free carboxylic and/or hydroxylic functional groups of the functionalized hyaluronic acid, or a derivative thereof.

Therefore, the crosslinked polymer of the present invention can comprise the following types of direct crosslinking (wherein the functionalized hyaluronic acid, or derivative thereof, is referred to, for practical purposes, as “HYD”):

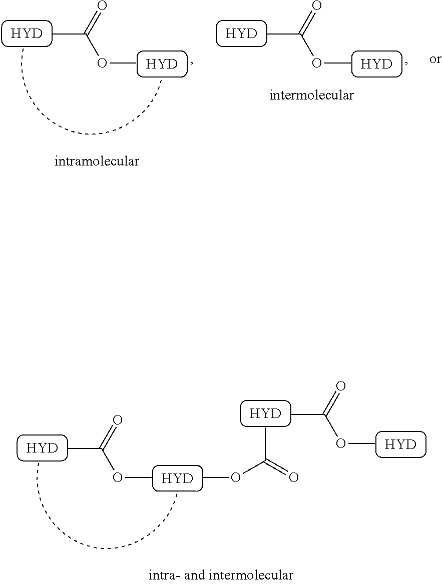

or of indirect crosslinking by spacer moiety (referred to, for practical purposes, as “SPC”):

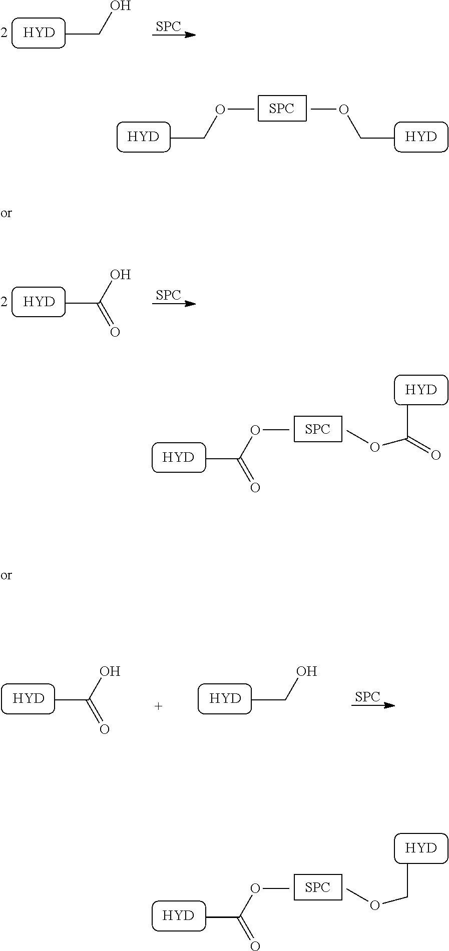

In some embodiments, said spacer moiety derives from bi- or polyfunctional epoxy selected from epichlorohydrin, 1,4-butanediol diglycidyl ether, 1,2-ethylenediol diglycidyl ether, 1-(2,3-epoxypropyl)-2,3-epoxycyclohexane, N,N-diglycidylaniline, epoxy-substituted pentaerythritol, and mixtures thereof.

Preferably, said spacer moiety derives from 1,4-butanediol diglycidyl ether. In this case, the crosslinked polymer of the present invention can comprise one or more of the following types of crosslinking:

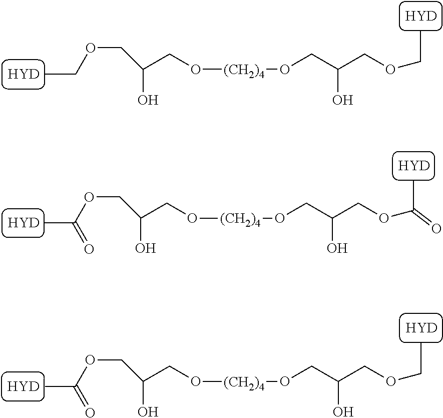

In other embodiments, said spacer moiety derives from divinyl sulfone. In this case, the crosslinked polymer of the present invention can comprise the following type of crosslinking:

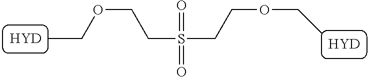

In other embodiments, said spacer moiety derives from a biscarbodiimide of formula Y₁—N═C═N—Y₂—N═C═N—Y₃, where Y₁ and Y₃ are, independently of each other, hydrogen, linear or branched aliphatic group C1-C10, alkoxy group C1-C10, cycloaliphatic group C1-C10, aryl C1-C10, heteroaryl C1-C10, aralkyl C1-C10, heteroaralkyl C1-C10, and Y₂ is a bifunctional moiety deriving from linear or branched aliphatic group C1-C10, alkoxy group C1-C10, cycloaliphatic group C1-C10, aryl C1-C10, heteroaryl C1-C10, aralkyl C1-C10, heteroaralkyl C1-C10. In this case, the crosslinked polymer of the present invention can comprise the following types of crosslinking:

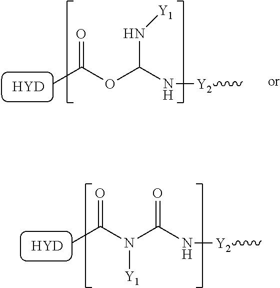

likewise the specular types of crosslinking on the imide function of the spacer moiety comprising the substituent Y₃.

Preferably, said biscarbodiimide is selected from 1,6-hexamethylene bis(ethylcarbodiimide), 1,8-octamethylene bis(ethylcarbodiimide), 1,10 decamethylene bis(ethylcarbodiimide), 1,12 dodecamethylene bis(ethylcarbodiimide), PEG-bis(propyl (ethylcarbodiimide)), 2,2′-dithioethyl bis(ethylcarbodiimide), 1,1′-dithio-p-phenylene bis(ethylcarbodiimide), para-phenylene-bis(ethylcarbodiimide), 1,1′-dithio-m-phenylene bis(ethylcarbodiimide) and mixtures thereof.

More preferably, said biscarbodiimide is para-phenylene-bis(ethylcarbodiimide). In a further aspect, the present invention also relates to a functionalized hyaluronic acid, or derivative thereof, comprising 10-90% of repeating units having the formula (I):

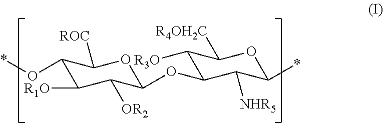

wherein
R₁, R₂, R₃, R₄ are, independently of one another, H, SO₃⁻, an acyl group derived from a carboxylic acid of the aliphatic, aromatic, arylaliphatic, cycloaliphatic, heterocyclic series, —CO—(CH₂)₂—COOY, where Y is a negative charge or H,
and
R is Z(1) or Z(2), and R₅ is —CO—CH₃, H, SO₃⁻, an acyl group derived from a carboxylic acid of the aliphatic, aromatic, arylaliphatic, cycloaliphatic, heterocyclic series, an acyl group of hyaluronic acid,
where Z(1) is a moiety of formula (1):

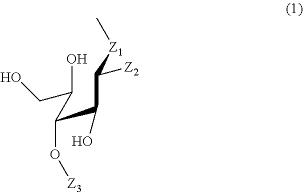

wherein Z₁ is —NR₆CH₂—, and R₆ is H or an aliphatic, aromatic, arylaliphatic, cycloaliphatic, heterocyclic group, substituted or non-substituted,
Z₂ is —OH, or —NHCOCH₃,
Z₃ is H, monosaccharide, disaccharide, or oligosaccharide, or Z(2) is a moiety of formula (2):

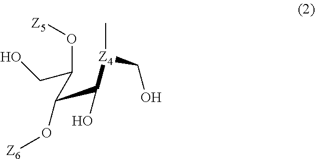

wherein Z₄ is —NR₆CH—, and R₆ is H or an aliphatic, aromatic, arylaliphatic, cycloaliphatic, heterocyclic group, substituted or non-substituted, Z₅ and Z₆ are, independently of each other, H, monosaccharide, disaccharide, or oligosaccharide,
   or
   R₅ is Z(3) or Z(4), and R is NR₆R₇, or an alcoholic group of the aliphatic, aromatic, arylaliphatic, cycloaliphatic, heterocyclic series, OH, O—, an alcoholic group of hyaluronic acid, an amino group of hyaluronic acid, and R₆, R₇ are, independently of each other, H or an aliphatic, aromatic, arylaliphatic, cycloaliphatic, heterocyclic group, substituted or non-substituted,
where Z(3) is a moiety of formula (3):

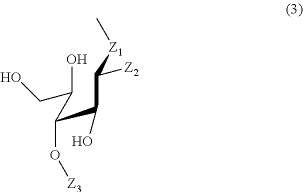

wherein Z₁ is —CH₂— or —CO—,
Z₂ is —OH, or —NHCOCH₃,
Z₃ is H, monosaccharide, disaccharide, or oligosaccharide, or Z(4) is a moiety of formula (4):

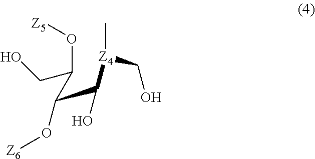

wherein Z₄ is —CH—,
Z₅ and Z₆ are, independently of each other, H, monosaccharide, disaccharide, or oligosaccharide,
   or

### R is Z(1) or Z(2), and R₅ is Z(3) or Z(4).

Preferably, said functionalized hyaluronic acid, or derivative thereof, comprises 10-60% of repeating units having the formula (I).

The term “aliphatic, aromatic, arylaliphatic, cycloaliphatic, heterocyclic” preferably means a moiety which is linear, branched, or cyclic, saturated or unsaturated, aliphatic or aromatic, selected from alkyl C1-C10, substituted alkyl C1-C10, alkenyl C2-C10, substituted alkenyl C2-C10, dienyl C4-C10, substituted dienyl C4-C10, alkynyl C2-C10, substituted alkynyl C2-C10, phenyl, substituted phenyl, aryl, substituted aryl, heteroaryl, substituted heteroaryl, alkylthio C1-C10, substituted alkylthio C1-C10, phenylthio, substituted phenylthio, arylthio, substituted arylthio, carbonyl, substituted carbonyl C1-C6, carboxyl, substituted carboxyl C1-C6, amino, substituted amino C1-C6, amide, substituted amide C1-C6, sulphonyl, substituted sulphonyl C1-C6, sulfonic acid, phosphonyl, substituted phosphonyl C1-C6, polyaryl, substituted polyaryl, cycloalkyl C3-C20, substituted cycloalkyl C3-C20, heterocycloalkyl C3-C20, substituted heterocycloalkyl C3-C20, cycloalkenyl C2-C10, substituted cycloalkenyl C2-C10, cyclodienyl C4-C10, substituted cyclodienyl C4-C10, or amino acid. The term “substituted” means linked to at least one halogen, hydroxyl, alkyl C1-C4, carboxyl, or combination thereof.

Preferably, Z₃, Z₅ and Z₆ are, independently of one another, H, moiety of glucose, galactose, arabinose, xylose, mannose, lactose, trehalose, gentiobiose, cellobiose, cellotriose, maltose, maltotriose, chitobiose, chitotriose, mannobiose, melibiose, fructose, N-acetyl glucosamine, N-acetyl galactosamine, or combination thereof.

More preferably, Z₃ is H, moiety of glucose, galactose, mannose, N-acetyl glucosamine, N-acetyl galactosamine, or a combination thereof.

In particularly preferred embodiments, the moiety of formula Z is a moiety of lactose or of galactose, where Z is anyone of Z(1), Z(2), Z(3) and Z(4).

In first embodiments, in 20-60% of the repeating units having the formula (I) present in the crosslinked polymer, R is Z(1) or Z(2), more preferably in 30-50%. Preferably, in these first embodiments, R₅ —COCH₃.

In second embodiments, in 5-30% of the repeating units having the formula (I) present in the crosslinked polymer, R₅ is Z(3) or Z(4), more preferably in 10-20%. Preferably, in these second embodiments, 10-20% of R₅ is Z(3) and 90-80% of R₅ is —COCH₃, while R is O—.

In further embodiments, the crosslinked polymer of the present invention comprises both repeating units having the formula (I) of said first embodiments and repeating units having the formula (I) of said second embodiments.

As can be seen from the structural formula shown above, hyaluronic acid, or derivative thereof, is functionalized through conjugation with a moiety of formula Z, the latter being Z(1), Z(2), Z(3) or Z(4), by:

1) an amide bond between the carboxylic group of the hyaluronic acid, or a derivative thereof, and an amine, via reductive amination of the precursor of Z with primary amines or ammonia sources,

2) an amine bond between the amine group of the hyaluronic acid, or a derivative thereof, which has already been deacetylated, and the moiety Z, via reductive amination,

3) an amide bond between the amine group of the hyaluronic acid, or a derivative thereof, which has already been deacetylated, and the carboxylic group of the precursor of the moiety Z.

Therefore, in a further aspect, the present invention concerns a process for the preparation of the functionalized hyaluronic acid, or derivative thereof, said process comprising the steps of:

i) providing hyaluronic acid, or a derivative thereof, which is partially or totally deacetylated;
ii) providing an amine derivative of a monosaccharide, disaccharide, oligosaccharide by means of reductive amination reaction;
iii) leaving the following to react:
a) said hyaluronic acid from step i) with the amine derivative from step ii) in the presence of carbodiimides and/or in the presence of carboxylic group activators,
   or
b) said partially or totally deacetylated derivative from step i) with a monosaccharide, disaccharide, oligosaccharide in the presence of an aminoborane;
   or
c) derivative partially or totally deacetylated from step i) with a derivative carboxylic of monosaccharide, disaccharide, oligosaccharide in the presence of carbodiimides and/or in the presence of carboxylic group activators;
   or
d) the derivative obtained in step iii-b) with the amine derivative from step ii) in the presence of carbodiimides and/or in the presence of carboxylic group activators;
   or
e) the derivative obtained in step iii-c) with the amine derivative from step ii) in the presence of carbodiimides and/or in the presence of carboxylic group activators;
   and
   iv) precipitating the functionalized hyaluronic acid, or derivative thereof, thus obtained with an organic solvent.

Surprisingly, it has been observed that the aminoboranes have a marked selectivity in the reduction of the imine group compared with the carbonyl group and are compatible with the aqueous environment allowing effective amine reduction of reducing sugars in the presence of primary amines, sources of ammonia and of amide residues of polysaccharides. At the same time, the presence of carbodiimides and or of carboxylic group activators effectively promotes the formation of amide derivatives of the hyaluronic acid with excellent selectivity compared with the formation of ester derivatives. Therefore, the process overall advantageously offers the possibility of conjugating monosaccharides, disaccharides and oligosaccharides on the main chain of hyaluronic acid without having recourse to the addition of chemical spacers.

The derivatives of hyaluronic acid which can be employed in the preparation of functionalized derivatives of the present invention are preferably the following:
hyaluronic acid salts, such as sodium hyaluronate, potassium hyaluronate, calcium hyaluronate, magnesium hyaluronate, zinc hyaluronate, cobalt hyaluronate, ammonium hyaluronate, tetrabutylammonium hyaluronate and mixtures thereof,
hyaluronic acid esters wherein a part or all the carboxyl groups are esterified with aliphatic, aromatic, arylaliphatic, cycloaliphatic, or heterocyclic series alcohols, as also described in EP0216453B1,
auto-crosslinked hyaluronic acid esters wherein a part or all the carboxyl groups are esterified with alcoholic groups from the same polysaccharidic chain or other chains, as also described in EP0341745B1,
crosslinked hyaluronic acid compounds wherein a part or all the carboxyl groups are esterified with aliphatic, aromatic, arylaliphatic, cycloaliphatic, or heterocyclic series polyalcohols, generating crosslinkings through spacer chains, as also described in EP0265116B1,
semi-esters of the succinic acid or heavy metal salts of succinic acid with hyaluronic acid or with partial or total hyaluronic acid esters, as also described in WO96/357207,
O-sulfated derivatives, as also described in WO95/25751, or N-sulfated derivatives, as also described in WO/1998/045335.

Said monosaccharide, disaccharide, or oligosaccharide corresponds to that defined above for the moiety Z.

Said amino-borane is preferably 2-methylpyridine borane, 5-ethyl-2-methylpyridine borane, pyridine borane, trimethylamine borane, triethylamine borane, dimethylamine borane, terz-butylamine borane, or a mixture thereof. More preferably, said amino-borane is 2-methylpyridine borane, 5-ethyl-2-methylpyridine borane, or a mixture thereof.

The aminoboranes can be employed in their natural state or already solubilised or dispersed in water-miscible organic solvents such as alcohols, and the most preferable of these are methanol, ethanol, 2-propanol, or a mixture thereof.

The term “organic solvent” means a water-miscible organic solvent which can lower the dielectric constant of the aqueous reaction solution. Suitable organic solvents are acetone, methanol, ethanol, 2-propanol, or a mixture thereof, preferably the organic solvent is ethanol or 2-propanol or a mixture thereof.

The term “carboxyl group activator” means those reagents which modify the hydroxyl function of said group, promoting the elimination thereof in the substitution reactions. Carboxylic group activators comprise hydroxybenzotriazole, 1,1′ -carbodiimidazole, p-nitrophenol, sodium salt of N-hydroxysulfosuccinimide, N-hydroxysuccinimide, and mixtures thereof.

Suitable carbodiimides comprise dicyclohexylcarbodiimide, 1-ethyl-3-(3-dimethylamino-propyl)carbodiimide hydrochloride, 1-ethyl-3-(3 -dimethylaminopropyl) carbodiimide, N,N′ -diisopropylcarbodiimide and mixtures thereof.

Optionally, the precipitate separated in step iv) is washed with mixtures of water and organic solvent, with percentages of water up to 30%, more preferably up to 10%.

Preferably, the molar ratio between the monosaccharide, disaccharide, or oligosaccharide from step iii) and the hyaluronic acid, or derivative thereof, is 0.5 to 30, more preferably 1 to 20, even more preferably 1 to 10.

In a still further aspect, the present invention concerns the use of the functionalized hyaluronic acid, or derivative thereof, as described above, for the preparation of the crosslinked polymer.

In this case, in a further aspect, the present invention concerns a process of preparation of the crosslinked polymer described above, comprising the steps of:
a) providing functionalized hyaluronic acid, or derivative thereof, as described above,
b) leaving the latter to react with a crosslinking agent selected from biscarbodiimides, or divinyl sulfone or an epoxy compound selected from aliphatic epoxies C2-C20, their halohydrines, epihalohydrines, and halides, or halides of methylpyridinium in the presence of a base, or a combination thereof, and
c) obtaining a crosslinked polymer gel.

In a further aspect, the present invention concerns the use of this crosslinked polymer in the treatment of disorders ascribable to altered galectin expression. Non-limiting examples of disorders concerned by over/under-regulation of these receptors are non-alcoholic steatohepatitis, plaque psoriasis, rheumatoid arthritis, osteoarthritis, neoplasms, adhesions, and dermal, pulmonary, renal, and cardiovascular fibrotic processes.

Examples of neoplasms and fibrotic processes are acute lymphoblastic leukaemia, idiopathic pulmonary fibrosis, hepatic fibrosis, cardiac fibrosis, renal fibrosis, and ovarian, prostate, lung, stomach, skin, thyroid, and pancreas cancers.

In a still further aspect, the present invention concerns the use of this crosslinked polymer as a biomaterial or scaffold for cellular growth, preferably in the treatment of orthopaedic disorders.

In an even further aspect, the present invention concerns the use of this crosslinked polymer as a biomaterial or scaffold for the cellular growth, in plastic/cosmetic surgery, haemodialysis, cardiology, angiology, ophthalmology, otorhinolaryngology, dentistry, gynaecology, urology, dermatology, oncology, and tissue repair.

The crosslinked polymer can also be employed as a biomaterial for coating objects used in both the medical field and in others sectors of industry, providing the surface of the object employed with new biological characteristics.

The objects which can be coated include, for example, catheters, cannulas, probes, heart valves, soft tissue prostheses, prostheses of animal origin, artificial tendons, bone and cardiovascular prostheses, contact lenses, artificial oxygenators for blood, kidneys, heart, pancreas, liver, blood bags, syringes, surgical instruments, filtration systems, laboratory instruments, containers for cultures and for the regeneration of cells and tissues, media for peptides, proteins and antibodies.

The crosslinked polymer can be used also in cosmetics and dermatology.

In a further aspect, the present invention regards a pharmaceutical composition comprising at least one crosslinked polymer and at least one pharmacologically active substance and/or at least one substance having, optionally, a biological function. Suitable pharmacologically active substances are antibiotics, anti-infectives, antimicrobials, antivirals, cytostatics, cytotoxics, anti-tumour agents, anti-inflammatory agents, cicatrizants, anaesthetics, analgesics, vasoconstrictors, cholinergic or adrenergic agonists and antagonists, antithrombotics, anticoagulants, haemostatics, fibrinolytics, thrombolytics, proteins and their fragments, peptides, polynucleotides, factors of growth, enzymes, vaccines, or a combination thereof.

Preferably, said substance having, optionally, a biological function is selected from collagen, fibrinogen, fibrin, alginic acid, sodium alginate, potassium alginate, magnesium alginate, cellulose, chondroitin sulfate, dermatan sulfate, keratan sulfate, heparin, heparan sulfate, laminin, fibronectin, elastin, polylactic acid, polyglycolic acid, poly(lactic-co-glycolic) acid, polycaprolactone, gelatine, albumin, poly(glycolide-co-caprolactone), poly(glycolide-co-trimethylene carbonate), hydroxyapatite, tricalcium phosphate, dicalcium phosphate, demineralised bone matrix and mixtures thereof. Preferably, said at least one crosslinked polymer and said at least one substance having, optionally, a biological function, have a weight ratio of 100:1 to 1:150.

In a still further aspect, the present invention concerns the use of this pharmaceutical composition in the treatment of disorders ascribable to altered galectin expression. Non-limiting examples of disorders concerned by over/under-regulation of these receptors are non-alcoholic steatohepatitis, plaque psoriasis, rheumatoid arthritis, osteoarthritis, neoplasms, adhesions, and dermal, pulmonary, renal, and cardiovascular fibrotic processes.

In an even further aspect, the present invention concerns the use of this pharmaceutical composition in reumatology, ortopedia, oncology, plastic/cosmetic surgery, haemodialysis, cardiology, angiology, oftalmology, otorhinolaryngology, dentistry, gynaecology, urology, oncology, dermatology and tissue repair.

Preferably, the pharmaceutical composition of the invention comprises up to 10 wt % of said at least one crosslinked polymer, based on the weight of the pharmaceutical composition, more preferably, up to 5 wt % of said at least one crosslinked polymer. Particularly preferable are the pharmaceutical compositions wherein said at least one crosslinked polymer amounts to 0.1-5 wt %, based on the weight of the composition.

In particularly preferred embodiments, the present invention regards a pharmaceutical composition comprising at least one crosslinked polymer, as described above, and hydroxyapatite, tricalcium phosphate or mixtures thereof. These compositions find advantageous use in orthopaedic applications concerning the skeletal system.

Said pharmaceutical composition can be administered by inhalation, by mouth, or by intramuscular, venous, intra-articular, transdermal, sub-cutaneous, or external or internal topical means, for example, surgically.

Preferably, said pharmaceutical composition is administered by intra-articular, sub-cutaneous, transdermal or topical means.

In some embodiments, the pharmaceutical composition is in a form which is injectable into the body's hard or soft tissues, such as organs, adipose, mucous membrane, gum, cartilage, and bone tissues, preferably by intradermal, subcutaneous, intramuscular, intra-articular or intraocular means.

In other embodiments, the pharmaceutical composition is for use in tissue repair or reconstruction, preferably in the creation or substitution of biological tissues or in the filling of biological tissues, such as cutaneous filling and filling of depressions, of bone cartilage or of joints.

In further embodiments, the pharmaceutical composition is for use in dermatological or cosmetic products, or for use as a medicine, preferably as a bio-resorbable implant.

The pharmaceutical composition can further comprise acceptable pharmaceutical excipients.

Suitable acceptable pharmaceutical excipients are for example pH regulators, isotone regulators, solvents, stabilisers, chelating agents, diluents, binding agents, disintegrants, lubricants, glidants, colorants, suspending agents, surfactants, cryoprotection agents, preservatives, and antioxidants.

The present invention regards furthermore a biomaterial comprising the crosslinked polymer, as described above, alone or in conjunction with at least one of the pharmacologically active and/or bioactive substances described above. Said biomaterial can be in the form of microspheres, nanospheres, membranes, sponge, wire, film, gauze, guide ways, pads, gel, hydrogels, fabrics, non-woven fabrics, cannulas, or a combination thereof.

It should be understood that all aspects identified as favourable and advantageous for the crosslinked polymer should be deemed likewise preferable and advantageous also for the functionalized hyaluronic acid, or derivative thereof, the preparation processes, the compositions, the biomaterials and the uses reported above.

It should furthermore be understood that all the possible combinations of the preferred aspects of the crosslinked polymer, functionalized hyaluronic acid, or derivative thereof, the preparation processes, the compositions, the biomaterials, and the uses disclosed above are likewise preferred.

Below are working examples of the present invention provided for illustrative purposes.

### EXAMPLES

### Example 1. Synthesis of Benzylamine Derivatives of Reducing Sugars

A solution of lactose (3% w/v), benzylamine (5% w/v) and 5-ethyl-2-methylpyridine borane (6% w/v) in water and methanol (3:1) was placed under agitation at a temperature of 55° C. and left to react for 20 hours. Next the mixture was cooled, extracted with dichloromethane and, finally, the aqueous phase was evaporated at low pressure obtaining a crystalline white solid which was then washed with diethyl ether and finally recovered by decantation and dried at reduced pressure. The product was characterised by IR and ¹H-NMR spectroscopy. Reaction yield: 90%.

### Example 2. Synthesis of Primary Amine Derivatives of Reducing Sugars

A solution of the derivative obtained in accordance with Example 2 (4% w/v) in methanol and water (1:1) was placed under magnetic agitation at room temperature. Next, Pd on carbon medium was added (0.4% w/v 10% on a metal medium) and the system thus produced pressurised with hydrogen. After 48 hours, the system was depressurised, additivated with an equi-volume of water, the decanted solid and the celite-filtered solution. The solution thus obtained was dried at reduced pressure providing a white solid. The product thus obtained was characterised by IR and ¹H -NMR spectroscopy. Reaction yield: 96%.

### Example 3. Synthesis of an Acylating Solution Based on an Imidazole Amide of Lactobionic Acid

A solution of lactobionic acid (10% w/v) in dimethyl sulfoxide was admixed with 1,1-carbodimidazole (1 eq.) and agitated at temperature for 2 hours. The solution thus produced was subsequently employed without further purification.

### Example 4. Synthesis of Partially Deacetylated Sodium Hyaluronate (48 h)

Sodium hyaluronate (2% w/v) was added to a solution of hydrazine sulfate (1% w/v) in hydrazine monohydrate and the system thus produced heated to 55° C. and left to react under agitation for 48 hours. Next, the raw reaction product was cooled, the product precipitated with ethanol, isolated, and washed with ethanol and dried for 24 hours at reduced pressure. Afterwards, the product obtained (5% w/v) was dissolved in an aqueous solution of acetic acid (5% V/V), the solution cooled to 4° C. and an aqueous solution of HIO₃ (0.5 M, 60% V/V) was added dropwise. The mixture was left to react in the same conditions for 1 h and then additivated with a solution of hydriodic acid (57% w/v, 11% V/V of the solution) and the system left to react for a further 15 minutes. The solution was then extracted with diethyl ether up to complete decolouration, the pH from aqueous phase adjusted to 7-7.5 with NaOH (1N, 0.1 N) and, to end, the product was precipitated with ethanol, washed with ethanol and dried. The product was characterised through ¹H-NMR and IR spectroscopy. Reaction yield: 83%, degree of de-acetylation: 11%.

### Example 5. Synthesis of Partially Deacetylated Sodium Hyaluronate (72 h)

A solution of sodium hyaluronate (2% w/v) and hydrazine sulfate (1% w/v) in hydrazine hydrate was placed under magnetic agitation at a temperature of 55° C. for 72 hours. At the end of the reaction time, ethanol was added to precipitate the polymer, the solid obtained was then washed with more ethanol and dried under a flow of nitrogen. The product was re-dissolved in a solution of aqueous acetic acid (6% w/v, 5% acetic acid), thermostatated at 0-5° C. and additivated with a volume (0.8 eq. in volume) of solution of iodic acid in water (7.5% w/v). The system thus produced was left under agitation for 1 hour, then additivated with a volume (0.11 eq. in volume) of aqueous hydriodic acid (57%) and left to react for a further 15 min. Next, the pH was brought to 9 through the addition of an aqueous solution of NaOH 1 M and the solution was extracted with diethyl ether up to complete decolouration. Afterwards, the product was precipitated with ethanol, washed with ethanol, desiccated at reduced pressure and characterised by IR and ¹H-NMR spectroscopy. Reaction yield: 86%, degree of de-acetylation: 20%.

### Example 6. Synthesis of Partially Deacetylated Sodium Hyaluronate (96 h)

Sodium hyaluronate (2% w/v) was added to a solution of hydrazine sulfate (1% w/v) in hydrazine monohydrate and the system thus produced heated to 55° C. and left to react under agitation for 96 hours. Next, the raw reaction product was cooled and the product was precipitated with ethanol, isolated, and washed with ethanol and dried for 24 hours at reduced pressure. Afterwards, the product obtained (5% w/v) was dissolved in an aqueous solution of acetic acid (5% V/V), the solution cooled to 4° C. and an aqueous solution of HIO₃ (0.5 M, 60% V/V) was added dropwise. The mixture was left in the same conditions to react for 1 h and then additivated with a solution of hydriodic acid (57% w/v, 11% V/V of the solution) and the system left to react for a further 15 minutes. The solution was then extracted with diethyl ether up to complete decolouration, the pH from the aqueous phase was adjusted to 7-7.5 with NaOH (1N, 0.1 N) and, to end, the product precipitated with ethanol, washed with ethanol, and dried. The product was characterised by IR and ¹H-NMR spectroscopy. Reaction yield: 86%, degree of de-acetylation: 21%.

### Example 7. Synthesis of Partially Deacetylated Sodium Hyaluronate (120 h)

Sodium hyaluronate (2% w/v) was added to solution of hydrazine sulfate (1% w/v) in hydrazine monohydrate and the system thus produced heated to 55° C. and left to react under agitation for 120 hours. Next, the raw reaction product was cooled and the product precipitated with ethanol, isolated, and washed with ethanol and dried for 24 hours at reduced pressure. Afterwards, the product obtained (5% w/v) was dissolved in an aqueous solution of acetic acid (5% V/V), the solution cooled to 4° C. and an aqueous solution of HIO₃ (0.5 M, 60% V/V) was added dropwise. The mixture was left in the same conditions to react for 1 h and then additivated with a solution of hydriodic acid (57% w/v, 11% V/V of the solution) and the system left to react for a further 15 minutes. The solution was then extracted with diethyl ether up to complete decolouration, the pH from the aqueous phase adjusted to 7-7.5 with NaOH (1N, 0.1 N) and, to end, the product precipitated with ethanol, washed with ethanol and dried. The product was characterised by IR and ¹H-NMR spectroscopy. Reaction yield: 89%, degree of de-acetylation: 26%.

### Example 8. Synthesis of Partially Deacetylated Sodium Hyaluronate (24 h)

A solution of sodium hyaluronate (2% w/v) and ammonium iodide (0.7% w/v) in hydrazine hydrate was placed under magnetic agitation at a temperature of 60° C. for 24 hours. At the end of the reaction time, ethanol was added to precipitate the polymer and the solid obtained was washed with ethanol and dried under a flow of nitrogen. The product was re-dissolved in a solution of aqueous acetic acid (6% w/v, 5% acetic acid), thermostatated at 0-5° C. and additivated with a volume (0.8 eq. in volume) of solution of iodic acid in water (7.5% w/v). The system thus produced was left under agitation for 1 hour, then additivated with a volume (0.11 eq. in volume) of aqueous hydriodic acid (57%) and left to react for a further 15 min. Next, the pH was brought to 9 through the addition of an aqueous solution of NaOH 1 M and the solution was extracted with diethyl ether up to complete decolouration. Afterwards, the product was precipitated with ethanol, washed with ethanol, and desiccated at reduced pressure. The solid thus obtained was characterised by IR and ¹H-NMR spectroscopy. Reaction yield: 88%, degree of de-acetylation: 15%.

### Example 9. Preparation of Tetrabutylammonium Salt of Hyaluronic Acid or Derivatives Thereof

An aqueous solution of sodium hyaluronate, or derivatives thereof, (1.6% w/v) was percolated through a column filled with sulfonic resin in the form of tetrabutylammonium salt (50% V/V of the solution) which had already been activated with a solution of tetrabutylammonium (40% w/v). The eluted solution was then lyophilised.

### Example 10. Amide Derivatives of Hyaluronic Acid (Amidation with Amine Derivatives of Reducing Sugars)

An aqueous solution, containing sodium hyaluronate (0.3% w/v), hydroxybenzotriazole (0.4% w/v), N-ethyl-N-(3-dimethylaminopropyl)carbodiimide hydrochloride (0.6% w/v) and the amine derivative of lactose obtained in accordance with Example 2 (2% w/v), was placed under agitation for 22 hours maintaining the pH at 6.8 through the addition of aqueous solutions of NaOH 0.1 M or of HCl 0.1 M. Next, NaCl (5 g/100 ml) was added and the product precipitated with methanol. The solid thus obtained was recovered by decantation and subjected to washing with methanol and water (4:1), and with pure methanol, and finally dried at reduced pressure. The product was characterised by IR and ¹H-NMR spectroscopy. Reaction yield: 86%, amidation with amine derivative of reducing sugar: 50%.

### Example 11. Amide Derivatives of the Hyaluronic Acid (Amidation with Amine Derivative of Reducing Sugars)

A solution of water and dioxane (1:1), containing sodium hyaluronate (0.5% w/v), N-hydroxysuccinimide (1.3% w/v), N-ethyl-N-(3-dimethylaminopropyl)carbodiimide hydrochloride (1.0% w/v) and the amine derivative of lactose obtained from Example 2 (2.1% w/v), was placed under agitation at room temperature for 12 hours. At the end of the reaction time, sodium hydrogen carbonate was added, taking the pH to approximately 9-10 and the solution was placed under agitation for a further 3 hours. The pH of the mixture was adjusted up to 7 through the addition of acetic acid (50%, V/V), and next sodium chloride (5 g/100 ml) was added and the product then precipitated with ethanol, washed with ethanol and with ether, and finally desiccated at reduced pressure. The product was characterised by IR and ¹H-NMR spectroscopy. Reaction yield: 85%, amidation with amine derivative of reducing sugar: 21%.

### Example 12. Amide Derivatives of the Hyaluronic Acid (Amidation with Amine Derivatives of Reducing Sugars) on an Organic Medium

A solution of salt of tetrabutylammonium of hyaluronic acid (2% w/v) in dimethyl sulfoxide was treated with aqueous hydrochloric acid up to pH 3 and then additivated with 1,1-carbonildiimidazole (1.5 eq.) and left to react for 12 hours. Next, the solution was filtered on a Gooch crucible to remove the solid moiety, the amine derivative obtained in Example 2 (2 eq.) was added, and the mixture thus produced left to react for 48 hours. Afterwards, a sodium chloride saturated solution was added in a sufficient volume to obtain a final titre of 5% w/v in sodium chloride, the mixture left under agitation for 1 hour and finally the product precipitated through the addition of acetone, the solid obtained was isolated and then dried. The product was characterised by IR and ¹H-NMR spectroscopy. Reaction yield: 80%, amidation with amine derivative of the reducing sugar: 10%.

### Example 13. Amide Derivatives of the Hyaluronic Acid (Amidation with Amine Derivatives of Reducing Sugars) on an Organic Medium

A solution of sodium hyaluronate (2% w/v) in dimethylformamide was admixed with 1,1-carbonildiimidazole (1 eq.). The solution thus produced was left to react for 6 hours, after which the amine derivative obtained in Example 2 (5 eq.) was added and the system left to react for a further 36 hours. Next, the product was precipitated with acetone then isolated, washed with acetone and dried at reduced pressure. The product was characterised by IR and ¹H-NMR spectroscopy. Reaction yield: 80%, amidation with amine derivative of the reducing sugar: 57%.

### Example 14. Amine Derivatives of Partially Deacetylated Hyaluronic Acid (Reductive Amination with Reducing Sugars)

An aqueous solution of partially deacetylated sodium hyaluronate obtained in accordance with Example 6 (1.5% w/v) was admixed with lactose (10 eq.) and the pH adjusted with acetic acid (100%) to reach values near 5.5. The system thus produced was heated to 60° C. and then additivated with a solution of 2-methylpyridine borane (10 eq., 10% w/v) in methanol and left to react for 2 hours in the same conditions. Next, the pH of the solution was adjusted with aqueous hydrochloric acid (4N) until values near 2-3 were reached and the system was maintained in the same conditions for 15 minutes, after which the system was cooled, the pH adjusted to 7-7.5 with NaOH (1 N) and the resulting solution dialysed repeatedly (cutoff 12-14000) against water. Finally, the solution was admixed with sodium chloride until the titre thereof reached 5% w/v and the desired product precipitated with ethanol, dried, and characterised by IR and ¹H -NMR spectroscopy. Reaction yield: 80%, amination with reducing sugar: 21%.

### Example 15. Amine Derivatives of Partially Deacetylated Hyaluronic Acid (Reductive Amination with Reducing Sugars)

An aqueous solution of partially deacetylated sodium hyaluronate obtained in accordance with Example 6 (1.5% w/v) was admixed with lactose (10 eq.) and the pH adjusted with acetic acid (100%) to reach values near 5.5. The system thus produced was heated to 60° C. and then additivated with a solution of 2-methylpyridine borane (10 eq., 10% w/v) in methanol and left to react for 2 hours in the same conditions. Next, the pH of the solution was adjusted with aqueous hydrochloric acid (4N) until values near 2-3 were reached and the system was maintained in the same conditions for 15 minutes. Afterwards, the system was cooled, the pH adjusted to 7-7.5 with NaOH (1 N) and sodium chloride until the titre thereof reached 5% w/v. The desired product was then precipitated with ethanol, dried, and characterised by IR and ¹H-NMR spectroscopy. Reaction yield: 84%, amination with reducing sugar: 21%.

### Example 16. Amine Derivatives of Partially Deacetylated Hyaluronic Acid (Reductive Amination with Reducing Sugars)

An aqueous solution of partially deacetylated sodium hyaluronate obtained in accordance with Example 4 (1.5% w/v) was admixed with lactose (10 eq.) and the pH adjusted with acetic acid (100%) to reach values near 5.5. The system thus produced was heated to 60° C. and then additivated with a solution of 2-methylpyridine borane (10 eq., 10% w/v) in methanol and left to react for 2 hours in the same conditions. Next, the pH of the solution was adjusted with aqueous hydrochloric acid (4N) until values near 2-3 were reached and the system was maintained in the same conditions for 15 minutes. Afterwards, the system was cooled, the pH adjusted to 7-7.5 with NaOH (1 N), and the resulting solution dialysed repeatedly (cutoff 12-14000) against water. Finally, the solution was admixed with sodium chloride until the titre thereof reached 5% w/v and the desired product precipitated with ethanol, dried, and characterised by IR and ¹H -NMR spectroscopy. Reaction yield: 78%, amination with reducing sugar: 11%.

### Example 17. Amine Derivatives of Partially Deacetylated Hyaluronic Acid (Reductive Amination with Reducing Sugars)

An aqueous solution of partially deacetylated sodium hyaluronate obtained in accordance with Example 5 (2% w/v) was admixed with lactose (3 eq.) and the pH was adjusted with acetic acid (100%) to reach values near 5.5. The system thus produced was heated to 60° C. and then additivated with a solution of 2-methylpyridine borane (1 eq., 10% w/v) in isopropanol and left to react for 3 hours in the same conditions. Next, the pH of the reaction was adjusted with aqueous hydrochloric acid (4 N) until values near 2-3 were reached and the system was maintained in the same conditions for 15 min. Afterwards, the system was cooled and the product was precipitated through the addition of isopropanol, washed with isopropanol and water (80:20 and 90:10), and dried at reduced pressure. The product was characterised by IR and ¹H-NMR spectroscopy. Reaction yield: 95%, amination with reducing sugar: 20%.

### Example 18. Amine Derivatives of Partially Deacetylated Hyaluronic Acid (Reductive Amination with Reducing Sugars)

An aqueous solution of partially deacetylated sodium hyaluronate obtained in accordance with Example 8 (2% w/v) was admixed with lactose (3 eq.) and the pH was adjusted with acetic acid (100%) to reach values near 5.5. The system thus produced was heated to 60° C. and then additivated with a solution of 2-methylpyridine borane (1 eq., 10% w/v) in isopropanol and left to react for 3 hours in the same conditions. Next, the pH of the reaction was adjusted with aqueous hydrochloric acid (4 N) until values near 2-3 were reached and the system was maintained in the same conditions for 15 min. Afterwards, the system was cooled and the product was precipitated through the addition of isopropanol, washed with isopropanol and water (80:20 and 90:10), and dried at reduced pressure. The product was characterised by IR and ¹H-NMR spectroscopy. Reaction yield: 95%, amination with reducing sugar: 15%.

### Example 19. Amide Derivatives of Compounds Obtained in Accordance with Examples 14-18 (Amidation of Derivatives Obtained Via Reductive Amination of Hyaluronic Acid with Reducing Sugars)

A solution of amine derivative of the hyaluronic acid obtained in accordance with Example 17 (0.25% w/v) in water was admixed with the amine derivative obtained in Example 1 (30 eq.) and the resulting solution brought to pH 6.8 through suitable addition of sodium hydroxide (1N, 0.1 N) or hydrochloric acid (1N, 0.1 N). Next, a solution was added dropwise, said solution being of (3-dimethylaminopropyl)-N′-ethylcarbodiimide hydrochloride (5 eq., 11% w/v) and hydroxybenzotriazole (3.5 eq., 6% w/v) which had already been solubilised in water:dimethyl sulfoxide (1.1:1). The pH of the solution was adjusted to 6.8 through suitable addition of sodium hydroxide (1N, 0.1 N) and the raw product thus produced left to react at room temperature for 16 hours. Next, the pH was appropriately brought to 7 with sodium hydroxide/hydrochloric acid (0.1 N) and the resulting solution dialysed repeatedly (cutoff 12-14000) against water. Afterwards, the solution was admixed with sodium chloride until the titre thereof reached 5% w/v and the desired product precipitated with ethanol, dried, and characterised by IR and ¹H-NMR spectroscopy. Reaction yield: 90%, amidation with amine derivative of the reducing sugar: 90%.

### Example 20. Amide Derivatives of Partially Deacetylated Hyaluronic Acid (Acylation with Carboxylic Derivatives of Reducing Sugars)

A solution of deacetylated sodium hyaluronate obtained in accordance with Example 6 (0.30% w/v) in water was admixed with lactobionic acid (30 eq.) and the resulting solution brought to pH 6.8 through suitable addition of sodium hydroxide (1N, 0.1 N) or hydrochloric acid (1N, 0.1 N). Next, a solution of (3-dimethylaminopropyl)-N′-ethylcarbodiimide hydrochloride (5 eq., 11% w/v) and hydroxybenzotriazole (3.5 eq., 6% w/v) which had already been solubilised in water:dimethyl sulfoxide (1.1:1) was added dropwise. The pH of the solution was adjusted to 6.8 through suitable addition of sodium hydroxide (1N, 0.1 N) and the raw product thus produced left to react at room temperature for 16 hours. Next, the pH was appropriately brought to 7 with sodium hydroxide/hydrochloric acid (0.1 N) and the resulting solution dialysed repeatedly (cutoff 12-14000) against water. Afterwards, the solution was admixed with sodium chloride until the titre thereof reached 5% w/v and the desired product precipitated with ethanol, dried, and characterised by IR and ¹H-NMR spectroscopy. Reaction yield: 79%, acylation with lactobionic acid: 5%.

### Example 21. Amide Derivatives of Partially Deacetylated Hyaluronic Acid (Acylation with Carboxylic Derivatives of Reducing Sugars)

A solution of lactobionic acid prepared in accordance with Example 3 was added to a solution of deacetylated sodium hyaluronate obtained in accordance with Example 6 (0.5 eq., 0.30% w/v) in water and the raw product thus produced left to react at room temperature for 16 hours. Next, the pH was appropriately brought to 7 with sodium hydroxide/hydrochloric acid (0.1 N) and the resulting solution dialysed repeatedly (cutoff 12-14000) against water. Afterwards, the solution was admixed with sodium chloride until the titre thereof reached 5% w/v and the desired product precipitated with ethanol, dried, and characterised by IR and ¹H-NMR spectroscopy. Reaction yield: 87%, acylation with lactobionic acid:16%.

### Example 22. Amide Derivatives of Partially Deacetylated Hyaluronic Acid (Acylation with Carboxylic Derivatives of Reducing Sugars)

A solution of lactobionic acid prepared in accordance with Example 3 was added to a solution of deacetylated sodium hyaluronate obtained in accordance with Example 6 (0.5 eq., 30% w/v) in water and the raw product thus produced left to react at room temperature for 16 hours. Next, a sodium chloride saturated solution was added in a sufficient volume to obtain a final titre of 5% w/v in sodium chloride, the mixture was left under agitation for 1 hour and finally the product precipitated through the addition of acetone; the solid obtained was isolated and then dried. The product was characterised by IR and ¹H-NMR spectroscopy. Reaction yield: 85%, acylation with lactobionic acid: 16%.

### Example 23. Amide Derivatives of Partially Deacetylated Hyaluronic Acid (Acylation with Carboxylic Derivatives of Reducing Sugars) on an Organic Medium

A solution of lactobionic acid prepared in accordance with Example 3 was added to a solution of deacetylated tetrabutylammonium hyaluronate obtained in accordance with Example 9 (0.5 eq., 2% w/v) in dimethyl sulfoxide and the raw product thus produced left to react at room temperature for 16 hours. Next, a sodium chloride saturated solution was added in a sufficient volume to obtain a final titre of 5% w/v in sodium chloride, the mixture left under agitation for 1 hour and finally the product precipitated through the addition of acetone; the solid obtained was isolated and then dried. The product was characterised by IR and ¹H-NMR spectroscopy. Reaction yield: 80%, acylation with lactobionic acid: 10%.

### Example 24. Amide Derivatives of Partially Deacetylated Hyaluronic Acid (Acylation with Carboxylic Derivatives of Reducing Sugars) on an Organic Medium

A solution of lactobionic acid prepared in accordance with Example 3 was added to a solution of deacetylated sodium hyaluronate obtained in accordance with Example 6 (0.5 eq., 2% w/v) in dimethylformamide and the raw product thus produced left to react at room temperature for 16 hours. Next, a sodium chloride saturated solution was added in a sufficient volume to obtain a final titre of 5% w/v in sodium chloride, the mixture left under agitation for 1 hour and finally the product precipitated through the addition of acetone, the solid obtained was isolated and then dried. The product was characterised by IR and ¹H-NMR spectroscopy. Reaction yield: 88%, acylation with lactobionic acid 19%.

### Example 25. Amide Derivatives of Compounds Obtained in Accordance with the Examples 20-24 (Amidation of Derivatives Obtained Via Acylation of the Hyaluronic Acid with Amine Derivatives of Reducing Sugars)

A solution of amide derivative of the hyaluronic acid obtained in accordance with the Example 24 (0.25% w/v) in water was admixed with the amine derivative obtained in

Example 1 (30 eq.) and the resulting solution brought to pH 6.8 through suitable addition of sodium hydroxide (1N, 0.1 N) or hydrochloric acid (1N, 0.1 N). Next, a solution of (3-dimethylaminopropyl)-N′-ethylcarbodiimide hydrochloride (5 eq., 11% w/v) and hydroxybenzotriazole (3.5 eq., 6% w/v) which had already been solubilised in water:dimethyl sulfoxide (1.1:1) was added dropwise. The pH of the solution was adjusted to 6.8 through suitable addition of sodium hydroxide (1N, 0.1 N) and the raw product thus produced left to react at room temperature for 16 hours. Next, the pH was appropriately brought to 7 with sodium hydroxide/hydrochloric acid (0.1 N) and the resulting solution dialysed repeatedly (cutoff 12-14000) against water. Afterwards, the solution was admixed with sodium chloride until the titre thereof reached 5% w/v and the desired product was precipitated with ethanol, dried, and characterised by IR and ¹H-NMR spectroscopy. Reaction yield: 84%, amidation with amine derivative of the reducing sugar: 93%.

### Example 26. Products of Crosslinking of Amide Derivatives of Hyaluronic Acid

Triethylamine (4% of the amide derivative of hyaluronic acid) was added to a solution (2.5%, w/v) of amide derivative of hyaluronic acid obtained in accordance with Examples 10 and 9 in dimethyl sulfoxide at 25° C. under agitation and the solution produced was agitated for a further 30 minutes. A solution of 2-chloro-1-methylpyridinium iodide (10.2% of the amide derivative of the hyaluronic acid) in dimethyl sulfoxide (1% w/v) was then added dropwise over a period of 20 minutes and the mixture thus produced agitated at 30° C. for a further 15 hours. Next, a solution of sodium chloride (2.5% w/v) amounting to 30% of the total volume was added gradually to the raw reaction product and the mixture formed transferred gradually into a volume of acetone amounting to approximately 200% of the volume reached. The precipitate formed was then isolated and washed 3 times with an acetone:water (5:1) mixture and once with just acetone. The product was then dried at reduced pressure for 24 hours at 30° C. and then stored at 4° C.

[50% amides, 25% esters, 25% sodium]

### Example 27. Products of Crosslinking of Amide Derivatives of Hyaluronic Acid

Triethylamine (1.6% of the amide derivative of the hyaluronic acid) was added to a solution (2.5%, w/v) of amide derivative of the hyaluronic acid obtained in accordance with examples 10 and 9 in dimethyl sulfoxide at 25° C. under agitation and the solution produced was agitated for a further 30 minutes. A solution of 2-chloro-1-methylpyridinium iodide (4.1% of the amide derivative of hyaluronic acid) in dimethyl sulfoxide (1% w/v) was then added dropwise over a period of 20 minutes and the mixture thus produced agitated at 30° C. for a further 15 hours. Next, a solution of sodium chloride (2.5% w/v) amounting to 30% of the total volume was added gradually to the raw reaction product and the mixture formed transferred gradually in a volume of acetone amounting to approximately 200% of the volume reached. The precipitate formed was then isolated and washed 3 times with an acetone:water (5:1) mixture and once with just acetone. The product was then dried at reduced pressure for 24 hours at 30° C. and then stored at 4° C.

[50% amides, 10% esters, 40% sodium]

### Example 28. Products of Crosslinking of Amine Derivatives of Hyaluronic Acid

Triethylamine (4% of the amide derivative of the hyaluronic acid) was added to a solution (2.5%, w/v) of amine derivative of the hyaluronic acid obtained in accordance with examples 15 and 9 in dimethyl sulfoxide at 25° C. under agitation and the solution produced was agitated for a further 30 minutes. A solution of 2-chloro-1-methylpyridinium iodide (10.2% of the amide derivative of the hyaluronic acid) in dimethyl sulfoxide (1% w/v) was then added dropwise over a period of 20 minutes and the mixture thus produced was agitated at 30° C. for a further 15 hours. Next, a solution of sodium chloride (2.5% w/v) amounting to 30% of the total volume was added gradually to the raw reaction product and the mixture formed transferred gradually in a volume of acetone amounting to approximately 200% of the volume reached. The precipitate formed was then isolated and washed 3 times with an acetone:water (5:1) mixture and once with just acetone. The product was then dried at reduced pressure for 24 hours at 30° C. and then stored at 4° C.

[30% esters, 70% sodium]

### Example 29. Products of crosslinking of amide derivatives of hyaluronic acid.

Triethylamine (4% of the amide derivative of the hyaluronic acid) was added to a solution (2.5%, w/v) of amine derivative of the hyaluronic acid obtained in accordance with examples 21 and 9 in dimethyl sulfoxide at 25 ° C. under agitation and the solution produced was agitated for a further 30 minutes. A solution of 2-chloro-1-methylpyridinium iodide (10.2% of the amide derivative of the hyaluronic acid) in dimethyl sulfoxide (1% w/v) was then added dropwise over a period of 20 minutes and the mixture thus produced agitated at 30° C. for a further 15 hours. Next, a solution of sodium chloride (2.5% w/v) amounting to 30% of the total volume was added gradually to the raw reaction product and the mixture formed transferred gradually in a volume of acetone amounting to approximately 200% of the volume reached. The precipitate formed was then isolated and washed 3 times with an acetone:water (5:1) mixture and once with just acetone. The product was then dried at reduced pressure for 24 hours at 30 ° C. and then stored at 4° C.

[30% esters, 70% sodium]

### Example 30. Products of crosslinking of amide derivatives of hyaluronic acid.

An aqueous solution (12% w/v) of amide derivative of hyaluronic acid obtained in accordance with example 10 was brought to pH 12-13 through the addition of aqueous NaOH (5 N). After 15 minutes of agitation at RT, 1,4-butandioldiglycidyletere (BDDE, 18% of the amide derivative of the hyaluronic acid) was added and the system placed under agitation at RT for 2 hours. Next the pH was brought to 7 with aqueous HCl (2N) and the system left in the same conditions for 15 hours. Next, the gel thus obtained was broken into pieces, washed with deionised water, refluxed for 8 hours in phosphate saline solution (1×), and then dried. Finally, the compound obtained was broken into pieces and stored at 8° C.

### Example 31. Products of Crosslinking of Amide Derivatives of Hyaluronic Acid

An aqueous solution (12% w/v) of amide derivative of hyaluronic acid obtained in accordance with Example 10 was brought to pH 3 through the addition of aqueous HCl (2 N). After 15 minutes of agitation at RT, 1,4-butandioldiglycidyletere (BDDE, 18% of the amide derivative of hyaluronic acid) was added and the system was placed under agitation at RT for 2 hours. Next, the pH was brought to 7 with aqueous NaOH (5N) and the system left in the same conditions for 15 hours. Next, the gel thus obtained was broken into pieces, washed with deionised water, refluxed for 8 hours in phosphate saline solution (1×), and then dried. Finally, the compound obtained was broken into pieces and stored at 8° C.

### Example 32. Products of Crosslinking of Amine Derivatives of the Hyaluronic Acid

An aqueous solution (12% w/v) of amine derivative of hyaluronic acid obtained in accordance with Example 15 was brought to pH 3 through the addition of aqueous HCl (2 N). After 15 minutes of agitation at RT, 1,4-butandioldiglycidyletere (BDDE, 18% of the amide derivative of hyaluronic acid) was added and the system placed under agitation at RT for 2 hours. Next, the pH was brought to 7 with aqueous NaOH (5N) and the system left in the same conditions for 15 hours. Next, the gel thus obtained was broken into pieces, washed with deionised water, refluxed for 8 hours in phosphate saline solution (1×), and then dried. Finally, the compound obtained was broken into pieces and stored at 8° C.

### Example 33. Products of Crosslinking of Amide Derivatives of Hyaluronic Acid

An aqueous solution (12% w/v) of amide derivative of hyaluronic acid obtained in accordance with Example 21 was brought to pH 3 through the addition of aqueous HCl (2 N). After 15 minutes of agitation at RT, 1,4-butandioldiglycidyletere (BDDE, 18% of the amide derivative of hyaluronic acid) was added and the system placed under agitation at RT for 2 hours. Next the pH was brought to 7 with aqueous NaOH (5N) and the system left in the same conditions for 15 hours. Next, the gel thus obtained was broken into pieces, washed with deionised water, refluxed for 8 hours in phosphate saline solution (1×), and then dried. Finally, the compound obtained was broken into pieces and stored at 8° C.

### Example 34. Products of Crosslinking of Amide Derivatives of Hyaluronic Acid

An aqueous solution (4% w/v) of amide derivative of hyaluronic acid obtained in accordance with Example 10 was brought to pH 12-13 through the addition of aqueous NaOH (5N) and agitated at room temperature for 30 minutes. Next, divinyl sulfone (DVS, 20% of the amide derivative of hyaluronic acid) was added gradually to the solution, leading to the formation of a gel in approximately 15 minutes. The gel formed was left in the same conditions for a further hour and then transferred into a volume of water amounting to 100 times the start volume. The gel was then left to swell for 15 hours and then crushed, washed repeatedly with water, and isolated in the form of transparent particles.

### Example 35. Products of Crosslinking of Amine Derivatives of Hyaluronic Acid

An aqueous solution (4% w/v) of amine derivative of hyaluronic acid obtained in accordance with Example 15 was brought to pH 12-13 through the addition of aqueous NaOH (5N) and agitated at room temperature for 30 minutes. Next, divinyl sulfone (DVS, 20% of the amide derivative of hyaluronic acid) was added gradually to the solution, leading to the formation of a gel in approximately 15 minutes. The gel formed was left in the same conditions for a further hour and then transferred into a volume of water amounting to 100 times the start volume. The gel was then left to swell for 15 hours and then crushed, washed repeatedly with water, and isolated in the form of transparent particles.

### Example 36. Products of Crosslinking of Amide Derivatives of the Hyaluronic Acid

An aqueous solution (4% w/v) of amide derivative of the hyaluronic acid obtained in accordance with example 21 was brought to pH 12-13 through the addition of aqueous NaOH (5N) and agitated at room temperature for 30 minutes. Next, divinyl sulfone (DVS, 20% of the amide derivative of the hyaluronic acid) was added gradually to the solution, leading to the formation of a gel in approximately 15 minutes. The gel formed was left in the same conditions for a further hour and then transferred into a volume of water amounting to 100 times the start volume. The gel was then left to swell for 15 hours and then crushed, washed repeatedly with water, and isolated in the form of transparent particles.

### Example 37. Products of Crosslinking of Amide Derivatives of the Hyaluronic Acid

A solution (1.5% w/v) of amide derivative of hyaluronic acid obtained in accordance with Example 10 in MES buffer (aqueous solution 1.5% w/v of 2-[N-morpholino]ethanesulfonic acid at pH 5.5) was admixed with p-phenylene-bis(ethylcarbodiimide) (20% of the amide derivative of the hyaluronic acid, in a 1.5% w/v acetone solution). The reaction mixture thus produced was agitated and then left at room temperature for a further 72 hours. Next, sodium chloride (325% of the amide derivative of the hyaluronic acid) was added and the gel left at room temperature for a further hour. The product of crosslinking was then precipitated by adding ethanol under vigorous agitation (900% v/v of the MES buffer), isolated, dried at reduced pressure, and then stored at 8° C.

### Example 38. Products of Crosslinking of Amine Derivatives of the Hyaluronic Acid

A solution (1.5% w/v) of amine derivative of hyaluronic acid obtained in accordance with Example 15 in MES buffer (aqueous solution 1.5% w/v of 2-[N-morpholino]ethanesulfonic acid at pH 5.5) was admixed with p-phenylene-bis(ethylcarbodiimide) (20% of the amide derivative of hyaluronic acid, in a 1.5% w/v acetone solution). The reaction mixture thus produced was agitated and then left at room temperature for a further 72 hours. Next, sodium chloride (325% of the amide derivative of hyaluronic acid) was added and the gel left at room temperature for a further hour. The product of crosslinking was then precipitated by adding ethanol under vigorous agitation (900% v/v of the MES buffer), isolated, dried at reduced pressure, and then stored at 8° C.

### Example 39. Products of Crosslinking of Amide Derivatives of the Hyaluronic Acid

A solution (1.5% w/v) of amide derivative of hyaluronic acid obtained in accordance with Example 21 in MES buffer (aqueous solution 1.5% w/v of 2-[N-morpholino]ethanesulfonic acid at pH 5.5) was admixed with p-phenylene-bis(ethylcarbodiimide) (20% of the amide derivative of hyaluronic acid, in a 1.5% w/v acetone solution). The reaction mixture thus produced was agitated and then left at room temperature for a further 72 hours. Next, sodium chloride (325% of the amide derivative of hyaluronic acid) was added and the gel left at room temperature for a further hour. The product of crosslinking was then precipitated by adding ethanol under vigorous agitation (900% v/v of the MES buffer), isolated, dried at reduced pressure, and then stored at 8° C.

1. A crosslinked polymer comprising a functionalized hyaluronic acid, or a derivative thereof, comprising 10-90% of repeating units having the formula (I) 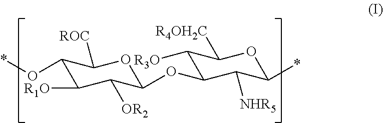
wherein
R₁, R₂, R₃, R₄ are, independently of one another, H, SO₃⁻, an acyl group derived from a carboxylic acid of the aliphatic, aromatic, arylaliphatic, cycloaliphatic, heterocyclic series, —CO—(CH₂)₂—COOY, where Y is a negative charge or H,
and
R is Z(1) or Z(2), and R₅ is —CO—CH₃, H, SO₃⁻, an acyl group derived from a carboxylic acid of the aliphatic, aromatic, arylaliphatic, cycloaliphatic, heterocyclic series, an acyl group of acid hyaluronic acid, where Z(1)is a moiety of formula (1): 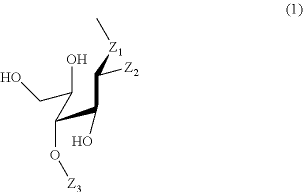
wherein Z₁ is —NR₆CH₂—, and R₆ is H or an aliphatic, aromatic, arylaliphatic, cycloaliphatic, heterocyclic group, substituted or non-substituted,
Z₂ is —OH, or —NHCOCH₃,
Z₃ is H, monosaccharide, disaccharide, or oligosaccharide,
or Z(2) is a moiety of formula(2): 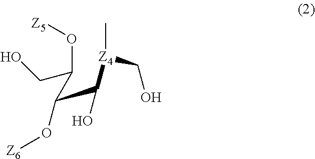
wherein Z ₄is —NR₆CH—, and R₆ is H or an aliphatic, aromatic, arylaliphatic, cycloaliphatic, heterocyclic group, substituted or non-substituted,
Z₅ and Z₆ are, independently of each other, H, monosaccharide, disaccharide, or oligosaccharide,
or
R₅ is Z(3) or Z(4), and R is NR₆R₇, or an alcoholic group of the aliphatic, aromatic, arylaliphatic, cycloaliphatic, heterocyclic series, OH, O⁻, an alcoholic group of hyaluronic acid, an amino group of hyaluronic acid, and R₆, R₇ are, independently of each other, H or an aliphatic, aromatic, arylaliphatic, cycloaliphatic, heterocyclic group, substituted or non-substituted,
where Z(3) is a moiety of formula (3): 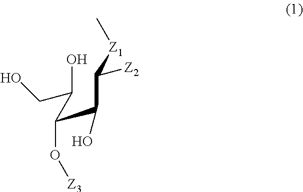
wherein Z₁ is —CH₂— or —CO—,
Z₂ is —OH, or —NHCOCH₃,
Z₃ is H, monosaccharide, disaccharide, or oligosaccharide,
or Z(4) is a moiety of formula (4): 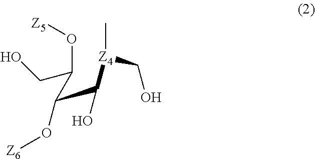
wherein Z₄ is —CH—,
Z₅ and Z₆ are, independently of each other, H, monosaccharide, disaccharide, or oligosaccharide,
or
R is Z(1) or Z(2), and R₅ is Z(3) or Z(4),
said functionalized hyaluronic acid, or a derivative thereof, being at least partially crosslinked directly by ester bond or lactone bond between carboxyl groups and hydroxyl groups of the same chain of functionalized hyaluronic acid, or a derivative thereof, and/or between carboxyl groups and hydroxyl groups of different chains, or
being at least partially crosslinked indirectly by a spacer moiety forming ester bonds with the carboxylic groups and/or ether bonds with the hydroxyl groups and/or amide bonds with the carboxyl groups, said spacer moiety being a biscarbodiimidic moiety or a bisvinylsulfonic moiety or an epoxy moiety deriving from bi- or polyfunctional epoxide selected from C2-C20 aliphatic epoxides, their halogenhydrons, epialogenhydrins, and halides, or a combination thereof

2. The crosslinked polymer of claim 1, wherein said functionalized hyaluronic acid, or a derivative thereof, comprises 10-60% of repeating units having the formula (I).

3. The crosslinked polymer of claim 1, wherein said bi- or polyfunctional epoxide is selected from epichlorohydrin, divinylsulfone, 1,4-butanediol diglycidyl ether, 1,2-ethylenediol diglycidyl ether, 1-(2,3-epoxypropyl)-2,3-epoxycyclohexane, N,N-diglycidylaniline, epoxy-substituted pentaerythritol, and mixtures thereof.

4. The crosslinked polymer of claim 1, wherein said biscarbodiimidic moiety derives from a biscarbodiimide of formula Y₁—N═C═N—Y₂—N═C═N—Y₃, where Y₁ and Y₃ are, independently of each other, hydrogen, linear or branched C1-C10 aliphatic group, C1-alkoxy group C10, cycloaliphatic group C1-C10, aryl C1-C10, heteroaryl C1-C10, aralkyl C1-C10, heteroaralkyl C1-C10, and Y₂ is a bifunctional moiety deriving from aliphatic C1-C10 linear or branched group, C1-C10 alkoxy group , cycloaliphatic group C1-C10, aryl C1-C10, heteroaryl C1-C10, aralkyl C1-C10, heteroaralkyl C1-C10.

5. The crosslinked polymer of claim 4, wherein said biscarbodiimidic moiety derives from a biscarbodiimide selected from 1,6-hexamethylene bis(ethylcarbodiimide), 1,8-octamethylene bis(ethylcarbodiimide), 1,10 decamethylene bis(ethylcarbodiimide), 1,12 dodecamethylene bis(ethylcarbodiimide), PEG-bis(propyl(ethylcarbodiimide)), 2,2′-dithioethyl bis(ethylcarbodiimide), 1,1′-dithio-p-phenylen bis(ethylcarbodiimide), para-phenylen-bis(ethylcarbodiimide), 1,1′-dithio-m-phenylen bis(ethylcarbodiimide) and mixtures thereof

6. Functionalized hyaluronic acid, or a derivative thereof, comprising 10-90% of repeating units having the formula (I): 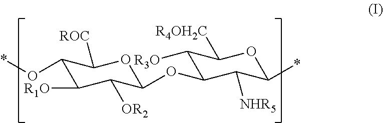
wherein
R₁, R₂, R₃, R₄ are, independently of one another, H, SO₃⁻, an acyl group derived from a carboxylic acid of the aliphatic, aromatic, arylaliphatic, cycloaliphatic, heterocyclic series, —CO—(CH₂)₂—COOY, where Y is a negative charge or H,
and
R is Z(1) or Z(2), and R₅ is —CO—CH₃, H, SO3⁻, an acyl group derived from a carboxylic acid of the aliphatic, aromatic, arylaliphatic, cycloaliphatic, heterocyclic series, an acyl group of acid hyaluronic acid,
where Z(1)is a moiety of formula (1): 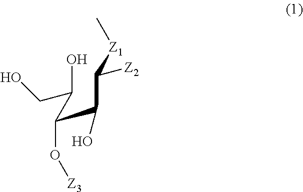
wherein Z₁ is —NR₆CH₂—, and R₆ is H or an aliphatic, aromatic, arylaliphatic, cycloaliphatic, heterocyclic group, substituted or non-substituted,
Z₂ is —OH, or —NHCOCH₃,
Z₃ is H, monosaccharide, disaccharide, or oligosaccharide,
or Z(2) is a moiety of formula(2): 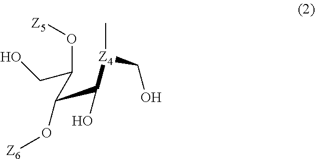
wherein Z₄ is —NR₆CH—, and R₆ is H or an aliphatic, aromatic, arylaliphatic, cycloaliphatic, heterocyclic group, substituted or non-substituted,
Z₅ and Z₆ are, independently of each other, H, monosaccharide, disaccharide, or oligosaccharide,
or
R₅ is Z(3) or Z(4), and R is NR₆R₇, or an alcoholic group of the aliphatic, aromatic, arylaliphatic, cycloaliphatic, heterocyclic series, OH, O⁻, an alcoholic group of hyaluronic acid, an amino group of hyaluronic acid, and R₆, R₇ are, independently of each other, H or an aliphatic, aromatic, arylaliphatic, cycloaliphatic, heterocyclic group, substituted or non-substituted,
where Z(3) is a moiety of formula (3): 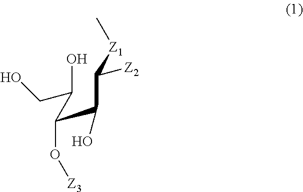
wherein Z₁ is —CH₂— or —CO—,
Z₂ is —OH, or —NHCOCH₃,
Z₃ is H, monosaccharide, disaccharide, or oligosaccharide,
or Z(4) is a moiety of formula (4): 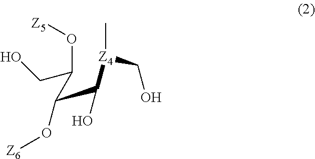
wherein Z₄ is -CH-,
Z₅ and Z₆are, independently of each other, H, monosaccharide, disaccharide, or oligosaccharide,
or
R is Z(1) or Z(2), and R₅ is Z(3) or Z(4).

7. The functionalized hyaluronic acid, or a derivative thereof, of claim 6, wherein Z₃, Z₅ and Z₆ are, independently of one another, H, moiety of glucose, galactose, arabinose, xylose, mannose, lactose, trealose, gentiobiose, cellobiose, cellotriose, maltose, maltotriose, chitobiose, chitotriose, mannobiose, melibiose, fructose, N-acetyl glucosamine, N-acetyl galactosamine, or a combination thereof.

8. The functionalized hyaluronic acid, or a derivative thereof, of claim 6, wherein Z₃ is H, moiety of glucose, galactose, mannose, N-acetyl glucosamine, N-acetyl galactosamine, or a combination thereof

9. The functionalized hyaluronic acid, or a derivative thereof, of claim 6, wherein Z is a moiety of lactose or galactose, where Z is any one of Z(1), Z(2), Z(3) and Z(4).

10. (canceled) .

11. A process for preparing the crosslinked polymer of claim 1, comprising the steps of:
a) providing a functionalized hyaluronic acid, or a derivative thereof, of claim 6,
b) reacting with a crosslinking agent selected from biscarbodiimides, or divinilsulfone, or an epoxy compound selected from C2-C20 aliphatic epoxides, their halogenhydrons, epialogenhydrins, and halides, or methylpyridinium halides in the presence of a base, or a combination thereof, and
c) obtaining a crosslinked polymer gel.

12. (canceled)

13. The crosslinked polymer of claim 1 to be used for use as a biomaterial or scaffold for cell growth.

14. A pharmaceutical composition comprising at least one crosslinked polymer of claim 1, and at least one pharmacologically active substance and/or at least one substance optionally having a biological function, wherein:
said pharmacologically active substance is selected from antibiotics, anti-infectives, antimicrobials, antivirals, cytostatic, cytotoxic, antitumor, anti-inflammatory, cicatrizant, anaesthetics, analgesics, vasoconstrictors, cholinergic or adrenergic agonists and antagonists, antithrombotic, anticoagulant, haemostatic, fibrinolytic, thrombolytic, proteins and fragments thereof, peptides, polynucleotides, growth factors, enzymes, vaccines, and combinations thereof, and
said substance optionally having a biological function is selected from collagen, fibrinogen, fibrin, alginic acid, sodium alginate, potassium alginate, magnesium alginate, cellulose, chondroitin sulfate, dermatan sulfate, keratan sulfate, heparin, eparan sulfate, laminin, fibronectin, elastin, polylactic acid, polyglycolic acid, poly(lactic-co-glycolic acid), polycaprolactone, gelatin, albumin, poly(glycolide-co-caprolactone), poly(glycolide-co-trimethylene carbonate), hydroxyapatite, tricalcium phosphate, dicalcium phosphate, demineralized bone matrix, and mixtures thereof.

15. A method of treating pathologies ascribable to an altered expression of galectins, said pathologies comprising non-alcoholic steatohepatitis, plaque psoriasis, rheumatoid arthritis, osteoarthritis, neoplasia, adhesions, and dermal, pulmonary, renal, and cardiovascular fibrotic processes, the method comprising administering to a subject in need thereof the crosslinked polymer of claim 1 or the pharmaceutical composition of claim 14.

16. The method of claim 15, wherein the pharmaceutical composition is in an injectable form suitable for hard or soft tissues of the body, such as organs, adipose tissues, mucous membranes, gums, cartilage and bones, preferably via intradermal, subcutaneous, intramuscular, intra-articular or intraocular route.

17. The method of claim 15, further comprising tissue repairing or reconstruction.

18. The method of claim 15, wherein the pharmaceutical composition is a dermatological or cosmetic product, or a medical device.

19. The method of claim 15, wherein said pathologies ascribable to an altered expression of galectins pertain to the medical field of for use in rheumatology, orthopedics, oncology, plastic-aesthetic surgery, hemodialysis, cardiology, angiology, ophthalmology, otorhinolaryngology, dentistry, gynecology, urology, dermatology, oncology, and tissue repair.

20. The method of claim 17, wherein said step comprising creating or replacing biological tissues or filling biological tissues comprising filling skin, depressions, bone cartilage or joint.

21. The method of claim 18, wherein said medical device is a bio-resorbable implant.
